(19) United States
(12) Patent Application Publication
(10) Pub. No.: US 2021/0345994 A1
(43) Pub. Date: Nov. 11, 2021

(54) Biological Sampling Apparatus

(71) Applicant: MS Pen Technologies, Incorporated, Tulsa, OK (US)

(72) Inventors: Shannon D. Green, Tulsa, OK (US); Aydin Zahedivash, Austin, TX (US)

(73) Assignee: MS Pen Technologies, Incorporated, Tulsa, OK (US)

(21) Appl. No.: 17/076,398

(22) Filed: Oct. 21, 2020

Related U.S. Application Data
(60) Provisional application No. 63/022,993, filed on May 11, 2020.

(51) Int. Cl. A61B 10/00

(52) U.S. Cl. CPC A61B 10/0051; A61B 2560/04

(57) ABSTRACT

In a general aspect, a sample is collected using a biological sampling apparatus. The biological sampling apparatus includes a shell having a closed end and an open end. The biological sampling apparatus also includes a harvesting tool comprising a shaft and a head. The shaft terminates in the head. The biological sampling apparatus additionally includes a translator configured to move the harvesting tool through the open end between a retracted position, where the head resides in the shell, and an extended position, where the head and at least a portion of the shaft reside outside the shell. A sealing member resides at the open end. The sealing member is configured to contact a subject surface and to create an enclosed sampling volume when collecting a specimen.

### CROSS-REFERENCE TO RELATED APPLICATIONS

This application claims priority to U.S. Provisional Application No. 63/022,993 filed May 11, 2020 and entitled “Biological Sampling Apparatus.” The priority application is hereby incorporated, in its entirety, by reference herein.

### BACKGROUND

The following description relates to a biological sampling apparatus.

Biological sampling is often used to collect samples from a target surface of a subject, for example, a nasal passage, the nasopharynx, the oropharynx, or the throat. The sample is then analyzed for the presence of organisms or other clinical markers for viral, bacterial, fungal or parasitic infections. As an example, a nasopharyngeal sampling is often used to collect nasal secretions from the nasopharynx, and the nasopharyngeal sample may then be analyzed to test for whooping cough, diphtheria, influenza, diseases (e.g., SARS, MERS, and COVID-19) caused by the coronavirus family of viruses, and others. As another example, a throat sampling is often used to collect saliva from the throat, and the throat sample may then be analyzed to test for bacteria or fungus that cause diseases such as strep throat, pneumonia, tonsillitis, and others.

### BRIEF DESCRIPTION OF THE DRAWINGS

FIG. 1A is schematic diagram of an example biological sampling apparatus having a sampling probe and a housing;

FIG. 1B is a schematic diagram of the example biological sampling apparatus of FIG. 1A, but in which the sampling probe is detached from the housing;

FIG. 2A is a schematic diagram of an example biological sampling apparatus having a sampling probe, an interface adapter, and a housing;

FIG. 2B is a detail view of the sampling probe of FIG. 2A;

FIG. 2C is a detail view of the interface adapter of FIG. 2A;

FIG. 2D is a schematic diagram of an alternate interface adapter for the example biological sampling apparatus of FIG. 2A;

FIG. 2E is a detail view of the housing of FIG. 2A;

FIG. 2F is a schematic diagram of an example nasal pillow adapter for the example biological sampling apparatus of FIG. 2A;

FIG. 2G is a schematic diagram of an example sampling procedure, shown in stages, for obtaining a biological sample from a surface of a subject;

FIG. 2H is a schematic diagram of an end of an example sampling probe and an example testing adapter configured to couple to the end of the example sampling probe;

FIG. 2I shows a top view of the example testing adapter of FIG. 2H;

FIG. 3A is a schematic diagram of an example sampling probe having a harvesting tool connected to a knob;

FIG. 3B is a schematic diagram of the example sample probe of FIG. 3A, but in which the harvesting tool is in an extended position;

FIG. 3C is a schematic diagram of the example sampling probe of FIG. 3A, but in which the example sample probe includes two snap rings;

FIG. 3D is a schematic diagram of the example sampling probe of FIG. 3A, but in which the example sample probe includes hinged lids at an open end;

FIG. 3E is a schematic diagram of the example sampling probe of FIG. 3D, but in which the harvesting tool is in a retracted position and the hinged lids are closed;

FIG. 4A is a schematic diagram of an example sampling probe having a multi-stage operation in which a harvesting tool rotates in a spiral motion;

FIG. 4B is a schematic diagram of the example sampling probe of FIG. 4A, but in which the harvesting tool is in an intermediate position between a retracted position and an extended position;

FIG. 4C is a schematic diagram of the example sampling probe of FIG. 4A, but in which the harvesting tool is in an extended position;

FIG. 5A is a schematic diagram of an example sampling probe having an adjustable extension length for a harvesting tool;

FIG. 5B is a schematic diagram of the example sampling probe of FIG. 5A, but in which the adjustable extension length of the harvesting tool is set for a child;

FIG. 6A is a schematic diagram of an example sampling probe that includes a bellow seal for articulating a harvesting tool and a plunger seal;

FIG. 6B is a schematic diagram of the example sampling probe of FIG. 6A, but in which the harvesting tool is in an extended position;

FIG. 7 is a schematic diagram of an example sampling probe that includes a bag seal and a plunger seal;

FIG. 8A is a schematic diagram of an example sampling probe that includes a multi-bar linkage coupled to a harvesting tool and a chamber;

FIG. 8B is a schematic diagram of the example sampling probe of FIG. 8A, but in which the harvesting tool is in an extended position;

FIG. 8C is a schematic diagram of the example sampling probe of FIG. 8A, but in which the harvesting tool is in an extended position and the chamber is breached by a sharp end of the harvesting tool;

FIG. 9A is a schematic diagram of an example sampling probe that includes a harvesting tool and a head of the harvesting tool that includes multiple fingers;

FIG. 9B is a schematic diagram of the example sampling probe of FIG. 9A, but in which the harvesting tool is in an intermediate position between a retracted position and an extended position;

FIG. 9C is a schematic diagram of the example sampling probe of FIG. 9A, but in which the harvesting tool is in an extended position;

FIG. 10A is a schematic diagram of an example head of a harvesting tool that includes a specimen acquisition surface and an array of specimen acquisition regions;

FIG. 10B is a schematic diagram of the example head of FIG. 10A, shown in cross-section, illustrating example specimen acquisition regions therein;

FIG. 10C is a detail view of one of the array of example specimen acquisition regions shown in FIG. 10A;

FIG. 10D is a schematic diagram, shown in cross-section, of an example containment chamber for the example specimen acquisition region of FIG. 10C;

FIG. 10E is a schematic diagram, shown in cross-section, of an example containment chamber for the example specimen acquisition region of FIG. 10C;

FIG. 10F is a schematic diagram, shown in cross-section, of an example containment chamber for the example specimen acquisition region of FIG. 10C; and

FIG. 10G is a schematic diagram, shown in cross-section, of the array of specimen acquisition regions shown in FIG. 10C, but in which the regions are fluidly coupled to an interior volume of a shaft of the harvesting tool.

### DETAILED DESCRIPTION

In some aspects of what is described here, a biological sampling apparatus includes a sampling probe and a housing. The sampling probe includes a harvesting tool, which can be used to obtain a sample from a target surface of a subject. The biological sampling apparatus may also include a translator, which can be used to extend the harvesting tool from a shell, so that a head of the harvesting tool can contact the target surface for sample collection. In some implementations, the harvesting tool extends from the shell and once the head of the harvesting tool reaches the target surface, the translator may provide a spiral motion to the harvesting tool to increase or maximize sample extraction. In some implementations, the biological sampling apparatus can include an interface that contacts the subject during the sampling process. For instance, the biological sampling apparatus may include a nasal pillow adaptor, which can form a comfortable contact and seal around the base of a nostril. The nasal pillow adaptor may have a pre-determined shape/geometry to guide the use of the sampling probe for self-administered home sample collection. After the sample is obtained, the translator may be used to retract the harvesting tool into the shell. The sampling probe may then be assembled with the housing, so that the sample can be fully contained, stored, or transported for testing.

In another instance, the biological sampling apparatus may include an oral adapter, which can form a comfortable contact and seal around a mouth. The oral adapter may have a pre-determined shape/geometry to guide the use of the sampling probe for self-administered home sample collection. After the sample is obtained, the translator may be used to retract the harvesting tool into the shell. The sampling probe may then be assembled with the housing, so that the sample can be fully contained, stored, or transported for testing.

In each of the examples shown and described here, the harvesting tool can be implemented as a swab or another type of harvesting tool. For instance, the harvesting tool can be a swab that includes a swab head supported by a swab shaft, and the swab may be of the type used to perform a nasal swab, nasopharyngeal swab, oropharyngeal swab, throat swab, or another type of swab procedure. Accordingly, the harvesting tools shown and described here may be used to collect nasal swab samples (containing material from a nasal passage), nasopharyngeal swab samples (containing material from the nasopharynx), oropharyngeal swab samples (containing material from the oropharynx), throat swab samples (containing material from the throat) or other types of swab samples collected from a target surface of a subject. In some instances, the harvesting tool may be used for collecting other types of liquid samples from other types of body cavities, such as lung, stomach, or other body cavities. In some instances, the biological sampling apparatus presented here can be adapted as an irrigation syringe, an applicator, or another type of system.

In some implementations, the biological sampling apparatus includes, or may function as, a sealed containment chamber to retain the sample in a controlled environment. For instance, the biological sampling apparatus can function as a sealed sample container for storing the sample, for transporting the sample between a sampling site and a testing site, etc. In some implementations, the biological sampling apparatus also includes an interface that couples with one or more testing instruments. For instance, the interface may allow a testing system to access the sample without exposing the sample to an external environment that could contaminate the sample or exposing the sample to personnel handling the device. For instance, the interface may include a membrane that seals the containment chamber, and the membrane can be punctured when the biological sampling apparatus interfaces with the testing system, to allow the testing system to directly access the sample.

In some examples, the techniques and apparatus described here may be adapted to perform sampling for bacterial infections, viral diseases, fungal infections, or other related pathogens and microorganisms. In some implementations, the techniques and apparatus described here may be adapted to perform sampling for whooping cough, diphtheria, influenza, diseases (e.g., SARS, MERS, and COVID-19) caused by the coronavirus family of viruses, strep throat, pneumonia, and tonsillitis. In some of the examples described here, a biological sampling apparatus can collect samples to be tested for COVID-19 or another type of coronavirus disease.

In some implementations, the techniques and apparatus disclosed here may provide technical advantages and improvements relative to conventional techniques. For example, the techniques and apparatus described here may retract and store a sample in a housing immediately after performing the sampling procedure, which can prevent the sample from being contaminated. In certain instances, the techniques and apparatuses described here can potentially reduce risks for healthcare professionals from being exposed to aerosols formed during a sampling procedure. In some instances, the techniques and apparatus described here can reduce biological risk for laboratory technicians from laboratory-acquired infections when handling pathogenic agents during testing, such as virus, parasites, fungi, rickettsia, or bacterial microorganisms. In some implementations, the number of steps required to perform a sampling procedure is reduced. Furthermore, the processes of sample collection, transportation and testing may be simplified. For example, viral transport media may be included in the sampling probe for specific types of tests. In some instances, the comfort of the patient during the sampling process is enhanced, for example, by a nasal pillow that forms a comfortable contact and seal around the base of a nostril. In some instances, the comfort of the patient is enhanced, for example, by a nasal pillow that forms a comfortable contact and seal around the base of a nostril. In some implementations, the techniques and apparatus described here can collect and retain a larger amount of specimen, improve sample yield and testing performance, e.g., sensitivity, accuracy and specificity. In some cases, a combination of these and potentially other advantages and improvements may be obtained.

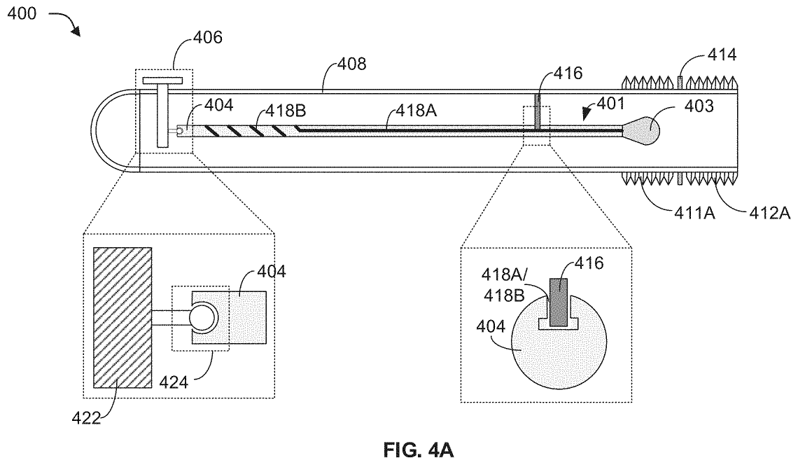

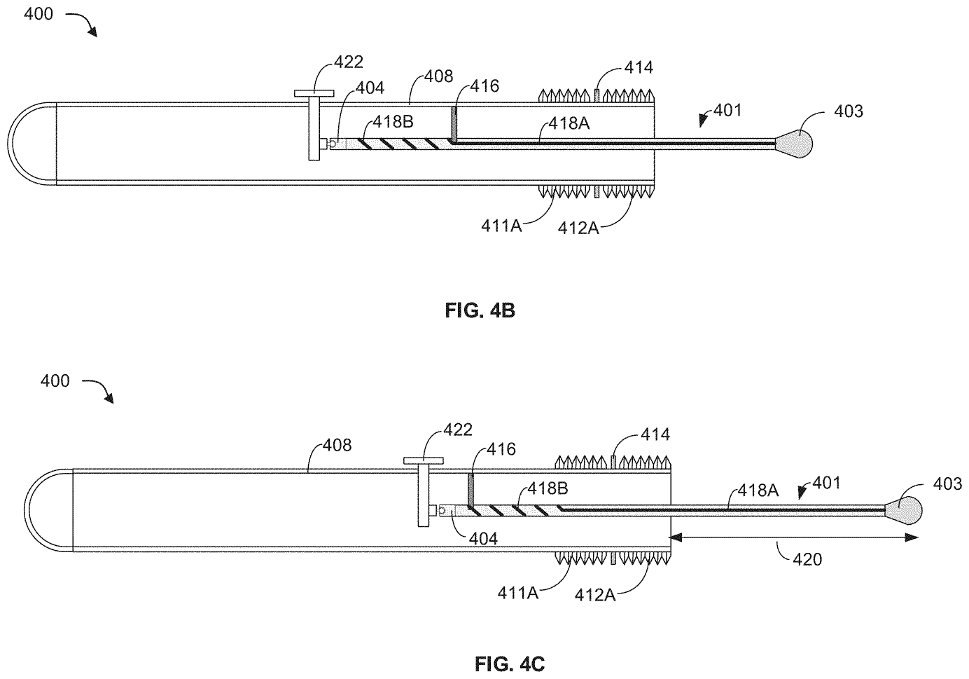

In some implementations, a sampling probe (e.g., the sampling probe 400 as shown in FIG. 4) includes an assembly. The assembly may include a knob and a joint linkage. In some instances, the surface of a shaft of a harvesting tool includes a first portion of a rail groove and a second portion of the rail groove. In some instances, the first and second portions of the rail groove on the shaft engage a track guide. In some implementations, the sampling probe may be configured to provide a two-step operation to the harvesting tool. For example, during a first step, a linear motion of the knob can provide a linear motion to the harvesting tool as the track guide moves along the first portion of the rail groove. During a second step, the linear motion of the knob is changed to a spiral motion of the harvesting tool as the track guide moves along the second portion of the rail groove. In some instances, the second portion of the rail groove may be designed to control the spiral motion of the harvesting tool during the second step.

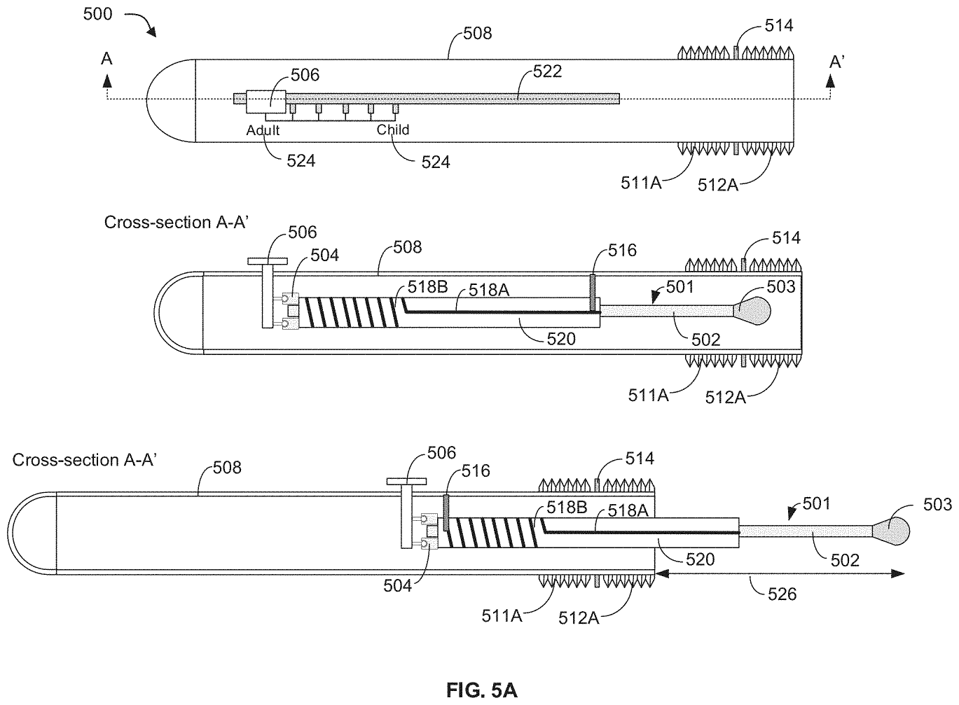

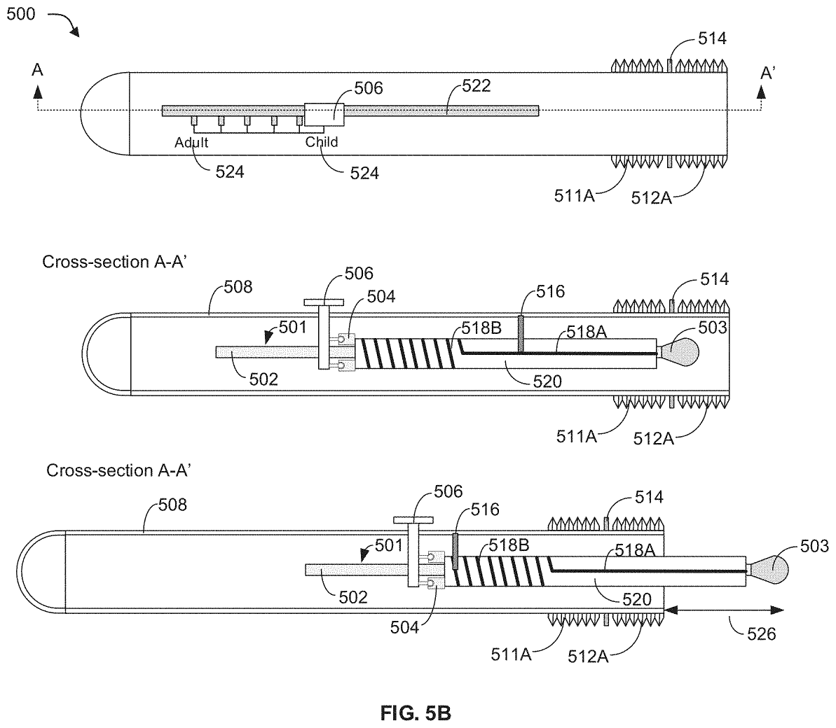

In some implementations, a sampling probe (e.g., the sampling probe 500 as shown in FIG. 5) can provide an adjustable length. For example, the sampling probe may include a sleeve, and the length may be adjusted by adjusting a position of the sleeve relative to the shaft of the harvesting tool. In some instances, a position of the sleeve relative to the shaft can be adjusted along the axial direction by adjusting a position of a knob that is mechanically connected to the sleeve. In some instances, the length may be adjusted according to the age group of a patient, e.g., a greater length for adults and a shorter length for children.

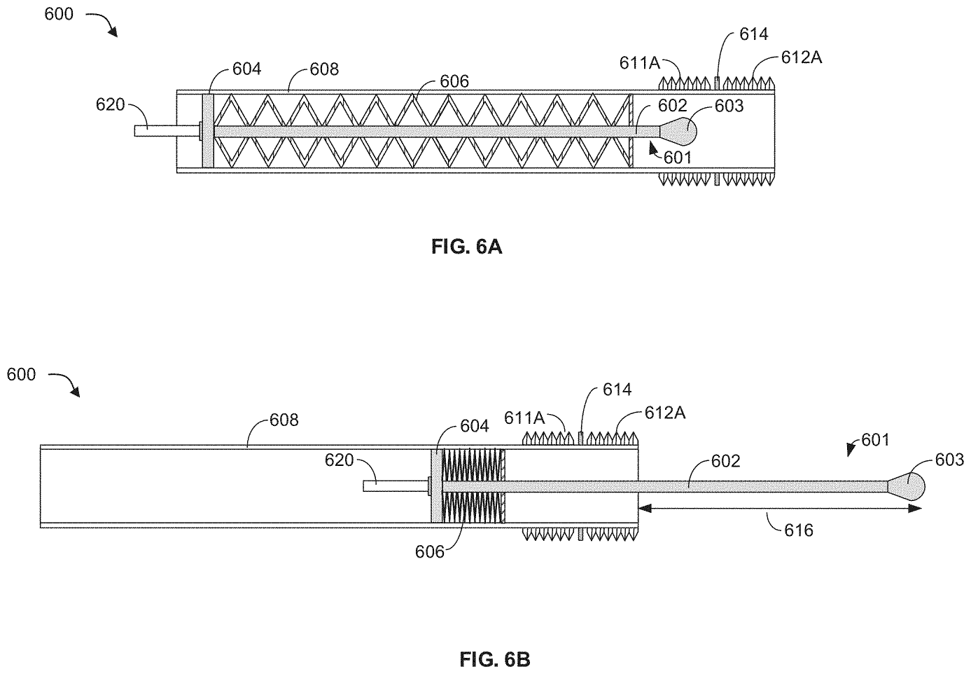

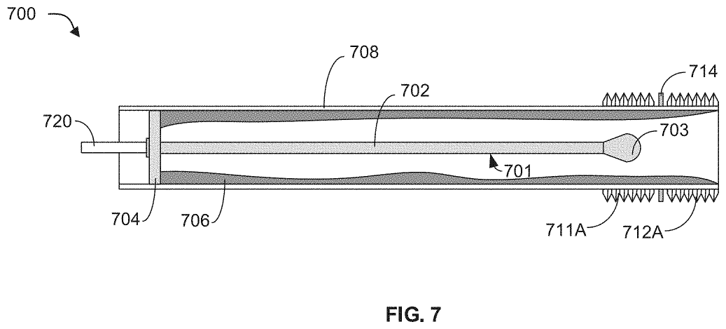

In some implementations, a sampling probe (e.g., the sampling probes 600, 700 as shown in FIGS. 6-7) may include a harvesting tool that is directly connected to a plunger seal. The sampling probe may include a plunger shaft to drive the plunger seal. In some instances, the plunger shaft is operated by a user to provide a linear motion to the plunger seal to extend or retract the harvesting tool. In some instances, the sampling probe may include a bellow seal (e.g., the bellow seal 606) or a bag seal (e.g., the bag seal 706) with one end anchored on an inner surface of the housing and one end on the plunger seal. In some instances, the bellow seal or the bag seal can provide an air-tight seal to the shell to prevent leakage or contamination. In some instances, the bellow seal or bag seal is not permeable to virus and can be composed of plastic, rubber or another type of material.

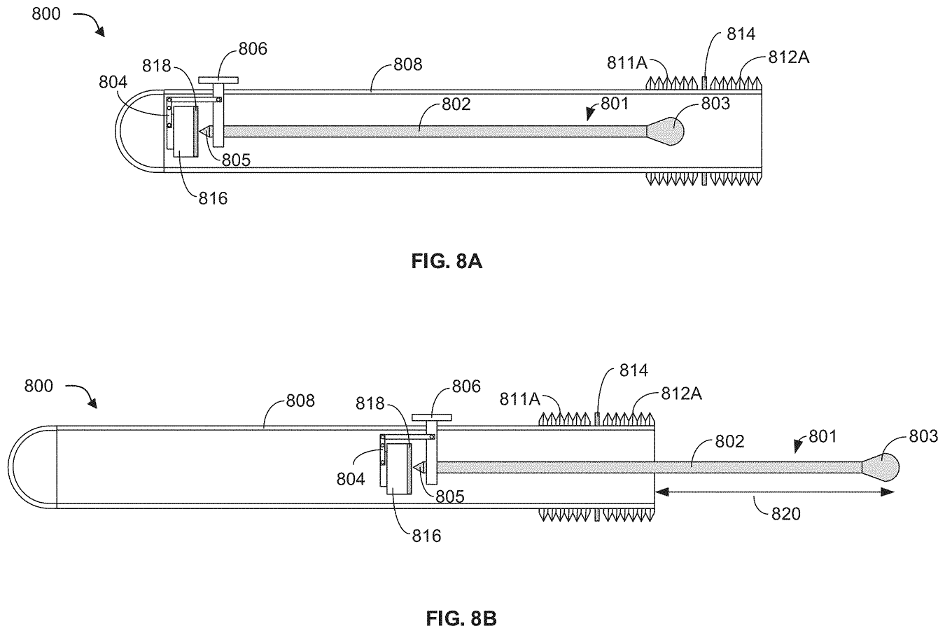

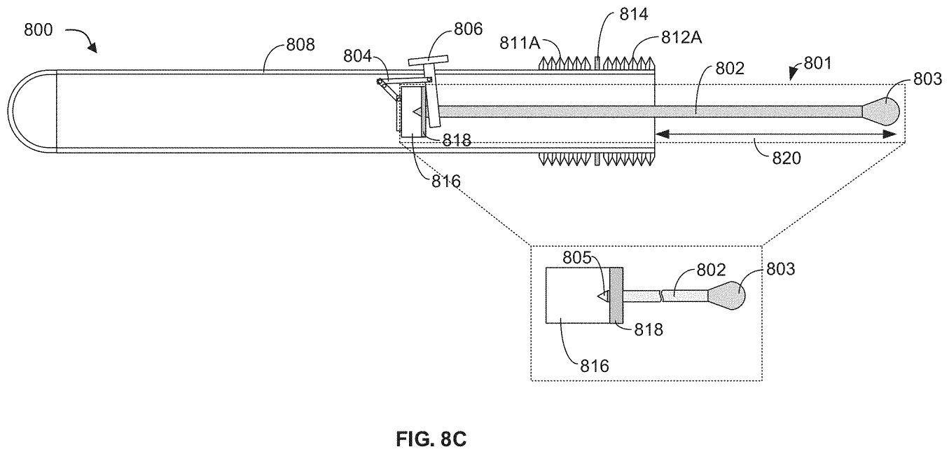

In some implementations, a sampling probe (e.g., the sampling probe 800 as shown in FIG. 8) may be configured for increasing or maximizing specimen collection. In some instances, the sampling probe may include a harvesting tool with a hollow shaft and a chamber. In some instances, the chamber may be a negative-pressure chamber sealed by a membrane (e.g., the membrane 818 in the chamber 816 shown in FIGS. 8A-8C). In some instances, the chamber may be pushed against one sharp end of the hollow shaft by a knob via a multi-bar linkage. In some instances, the low pressure in the chamber may facilitate and maximize an amount of specimen drawn from the target surface of the subject.

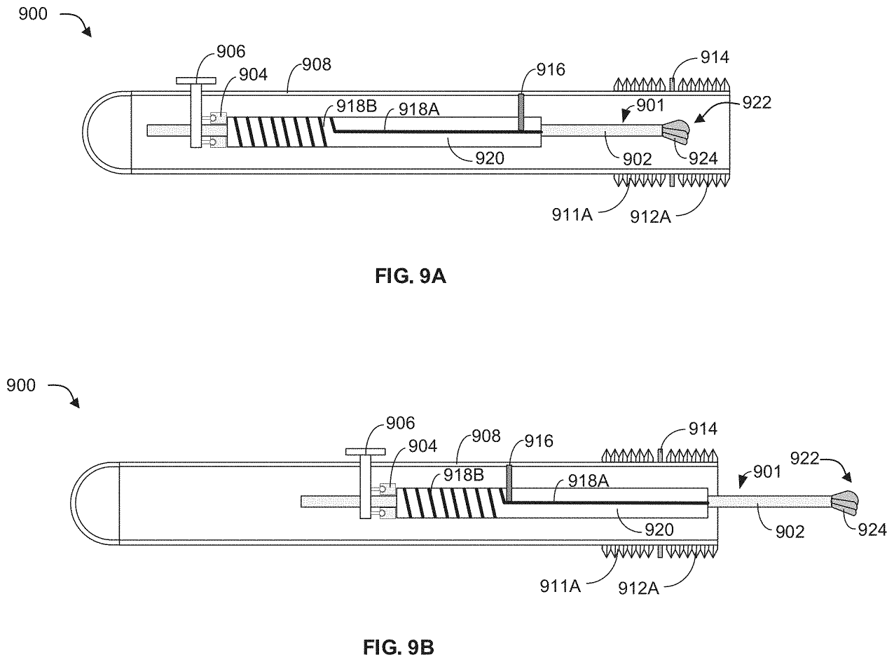

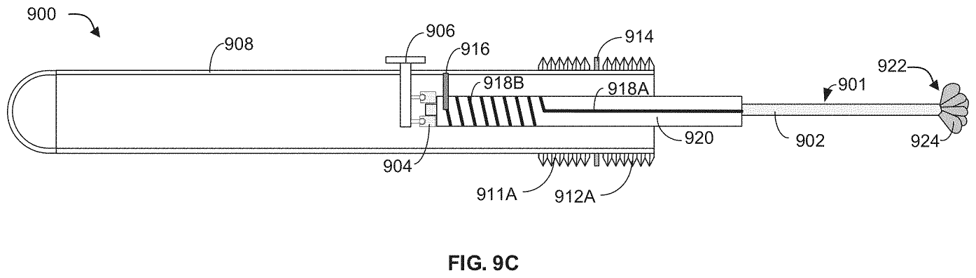

In some implementations, the sampling probe (e.g., the sampling probe 900 as shown in FIG. 9) includes a harvesting tool with a head that includes multiple fingers, e.g., the head 922 with fingers 924 as shown in FIGS. 9A-9C. In some instances, each of the fingers may extend along the radial direction while rotating along the axial direction. In certain examples, the radially extended fingers with a spiral motion may provide additional contact for an increased extraction of cells, molecules, or microorganism from the target surface of the subject. In some instances, the fingers may return to their original position from the radially extended position when the harvesting tool is extracted back into the housing after the sampling procedure.

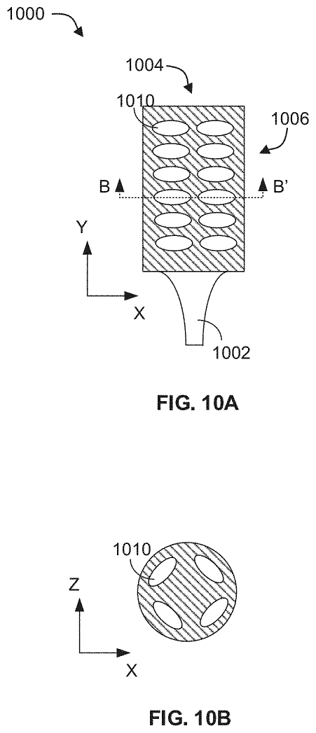

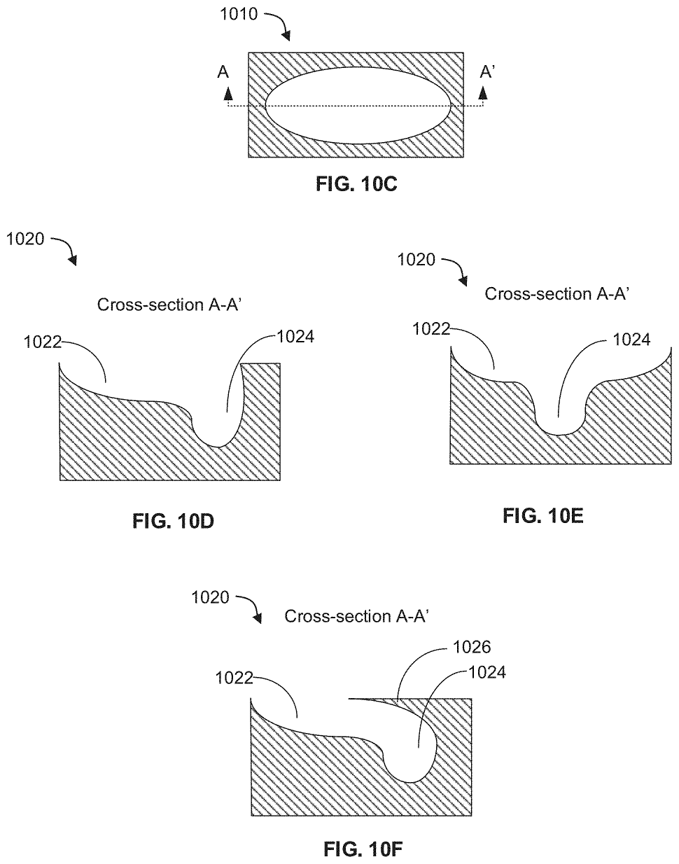

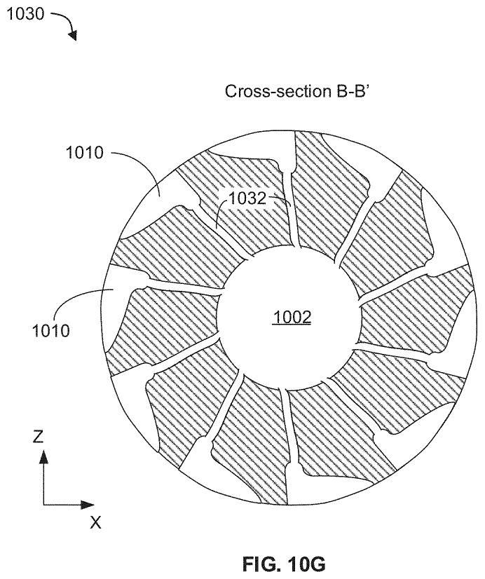

In some implementations, a sampling probe includes a head that has a contoured specimen acquisition surface (e.g., the harvesting tool 1000 shown in FIG. 10). For instance, the specimen acquisition surface may include an array of specimen acquisition regions, where each specimen acquisition region is configured to acquire and retain a liquid sample volume. Each of the specimen acquisition regions may be fluidically isolated from neighboring specimen acquisition regions. Each of the specimen acquisition regions may include a containment chamber. In some instances, the containment chambers may each include a shallow region and a deep region. In some instances, each of the specimen acquisition regions is fluidically coupled with a hollow shaft via respective channels. During operation, the liquid sample from the target surface may be collected into each of the specimen acquisition regions and extracted via the respective channels to the hollow shaft.

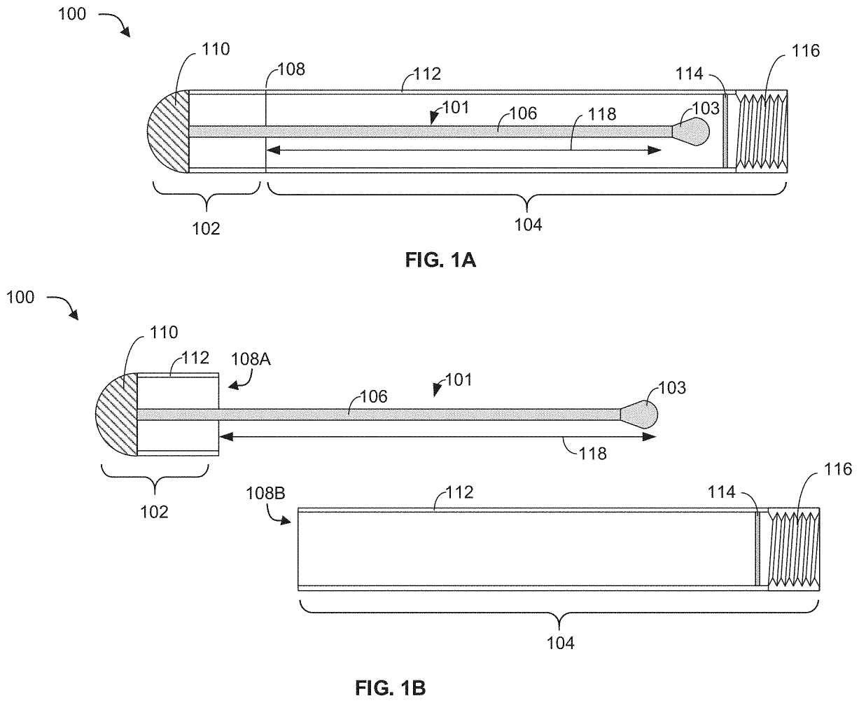

FIGS. 1A-1B are schematic diagrams showing aspects of an example biological sampling apparatus 100. In some instances, the example apparatus 100 can be used for sampling (e.g., collecting a biological specimen with a harvesting tool) to obtain a sample. In some implementations, the example apparatus 100 can be used as a sealed sample vial for storing the sample after specimen collection and during transportation between a sampling site and a testing site. In some instances, the example apparatus 100 provides an interface for direct coupling with a testing instrument, so that the sample may be extracted for testing without reopening the apparatus 100, thereby reducing potential contamination to the sample. As shown in FIG. 1, the example apparatus 100 includes a sampling probe 102 and a housing 104. In some examples, the example apparatus may include additional or different components, and the components may be arranged as shown or in another manner.

As shown in FIG. 1, the sampling probe 102 includes a harvesting tool 101, a base member 110 and a first portion of a tubular member 112. The harvesting tool 101 includes a head 103 and a shaft 106. In some instances, one end of the shaft 106 may be directly connected/anchored at the base member 110. In some implementations, the first portion of the tubular member 112 on the sampling probe 102 provides a grip. The grip can be used, for example, to operate the harvesting tool 101 as a handheld sampling probe, for specimen collection to obtain a sample. In some instances, operating the harvesting tool 101 for specimen collection may include performing a nasal sampling, nasopharyngeal sampling, oropharyngeal sampling, throat sampling, or another type of sampling procedure. The samples that can be obtained by the sampling probe 102 may include nasal samples (containing material from a nasal passage), nasopharyngeal samples (containing material from the nasopharynx), oropharyngeal samples (containing material from the oropharynx), throat samples (containing material from the throat) or other types of samples collected from a target surface of a subject.

In some instances, the harvesting tool 101 may be disconnected from the base member 110 and a new harvesting tool 101 may be installed. In some instances, a length 118 from one end of the first portion of the tubular member 112 to one end of the head 103 is in a range of 15-18 cm or in another range according to the types of samples to be collected. In some instances, the harvesting tool 101 may include a polyester head, a woven microfiber head, a cotton head, or another head made of another material. In some instances, the harvesting tool 101 may be implemented as the harvesting tool 1000 as shown in FIG. 10 or another type of harvesting tool that has other types of features and components.

As shown in FIG. 1, the housing 104 includes a second portion of the tubular member 112, a membrane 114 and an interface 116. In some instances, the harvesting tool 101 is completely enclosed by the second portion of the tubular member 112. The second portion of the tubular member 112 (of the housing 104) may be connected to or disconnected from the first portion of the tubular member 112 (of the sampling probe 102). As shown, the two portions of the tubular member 112 can be connected by mating the joints 108, which include a first joint 108A on the sampling probe 102 and a second joint 108B on the housing 104. For example, the joints 108 may include a screw connector, a snap-ring connector, or another type of integral joint or fastener. In certain examples, mating the joints 108 provides an air-tight seal to isolate the harvesting tool 101 inside the apparatus 100 from the surrounding environment.

In some instances, the interface 116 on one end of the housing 104 (the end opposite the end of the joint 108B of the housing 104), is used for directly coupling with one or more testing instruments. In some cases, the interface 116 can be configured as a universal interface that can couple to a variety of testing instruments, for example, by coupling to a port, inlet, or another type of complementary interface of the testing instrument. In some cases, the interface 116 can couple with one or more of mass spectrometers, liquid chromatography systems, instruments for genetic assays, biochemical analyses, electromagnetic analyses, and possibly others. In certain instances, the interface 116 may be implemented as an internal thread (threading on the internal surface of the tubular member 112), an external thread (threading on the external surface of the tubular member 112), a snap ring, or in another type of integral joint or fastener. In some instances, the interface 116 of the example apparatus 100 is configured to receive a probe or connect to a port of the testing instrument, which can break the seal by, for example, puncturing the membrane 114. In some instances, the specimen from the head 103 may be extracted to the testing instrument for testing without reopening the apparatus 100. In some instances, the membrane 114 of the housing 104, when assembled with the sampling probe 102, can isolate the sample from the surrounding environment. In some instances, the membrane 114 can also provide a seal against the inserted probe to prevent leakage during testing. In some instances, the specimen from the sample may be extracted and directed to the testing instrument through the probe coupled to the interface 116 or in another manner (e.g., according to the type of testing instrument). In some implementations, the housing 104 may include materials such as plastic, metal, glass, or another material. In some instances, the membrane 114 may include silicone, or another type of material.

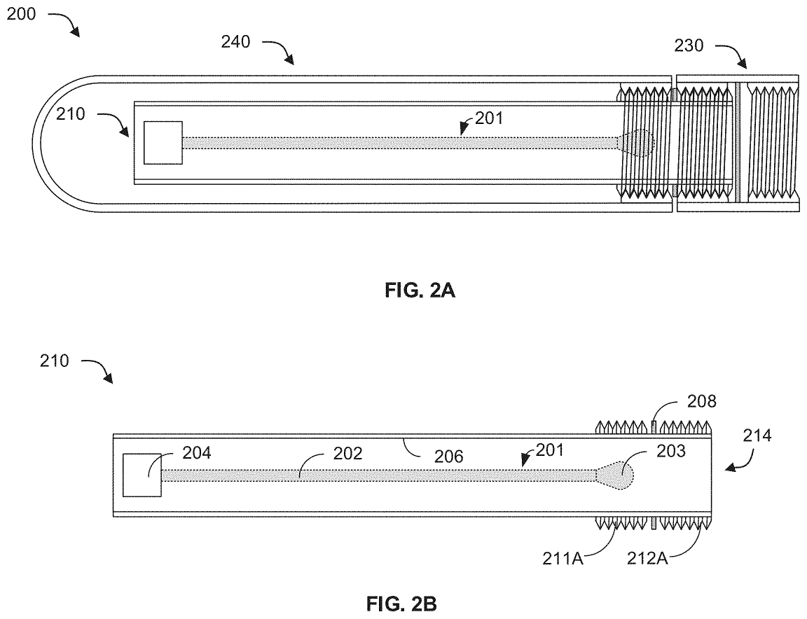

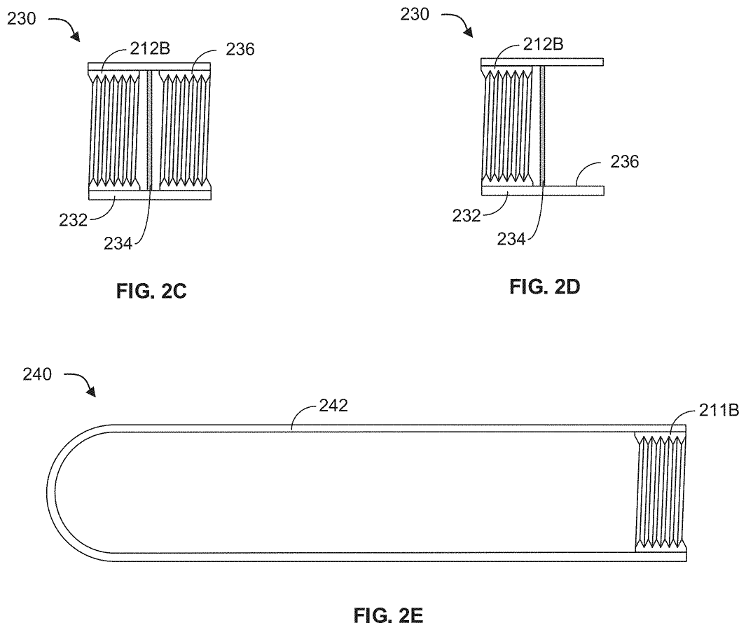

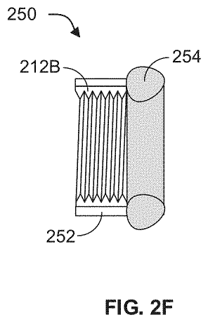

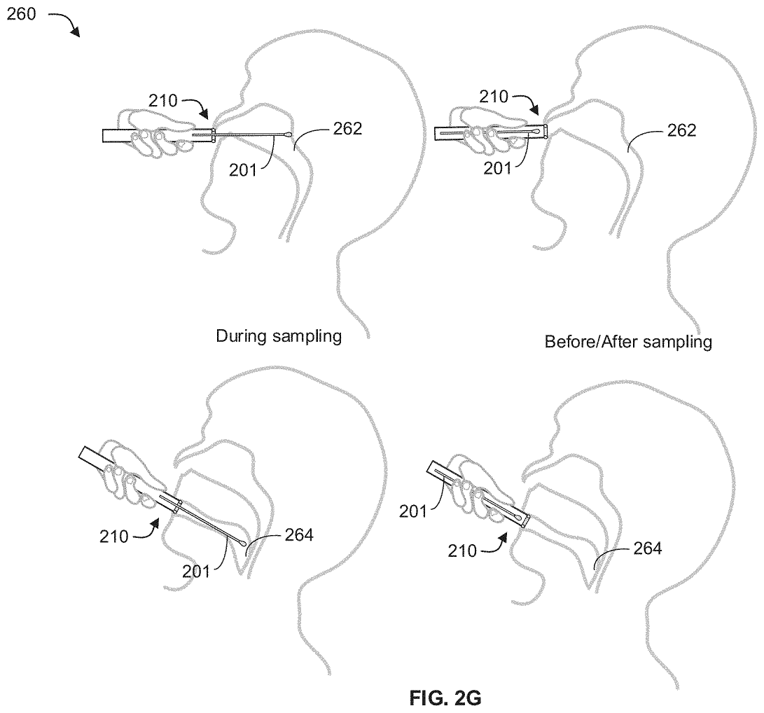

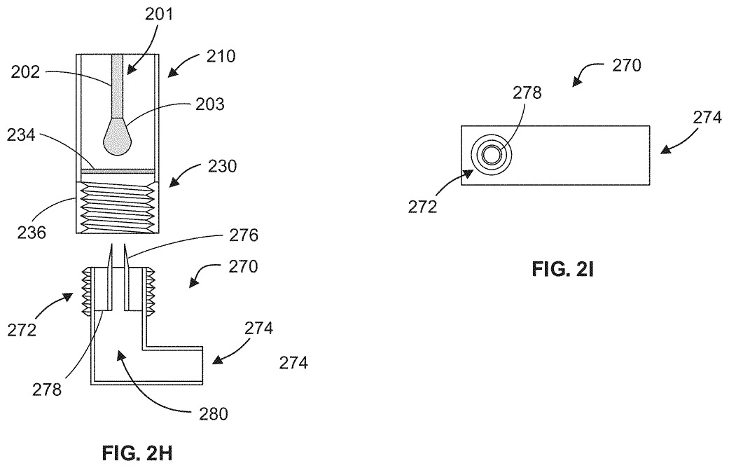

FIGS. 2A-2F are schematic diagrams showing aspects of an example biological sampling apparatus 200. In some instances, the example apparatus 200 can be used for sampling (e.g., collecting a biological specimen with a harvesting tool) to obtain a sample. In some implementations, the example apparatus 200 can be used as a sealed sample vial for storing the sample after specimen collection and during transportation between a sampling site and a testing site. In some instances, the example apparatus 200 provides an interface that can be directly connected with a testing instrument, so that the sample can be extracted for testing. In some instances, the sample in the example apparatus 200 may be tested without reopening the apparatus 200, which can provide facile operation and reduce potential contamination to the sample. The example apparatus 200 may also reduce risks to healthcare professionals, for example, reducing their exposure to aerosols or leaked fluids formed during a sampling procedure. As shown in FIGS. 2A-2F, the example apparatus 200 may include one or more of a sampling probe 210, an interface adaptor 230, a housing 240, and a nasal pillow adaptor 250. The example apparatus 200 may be assembled as shown in FIG. 2A or in another manner. In some examples, the example apparatus 200 may include additional or different components, and the components may be arranged as shown or in another manner.

The example sampling probe 210 shown in FIG. 2A includes a tubular member 206, a translator 204 and a harvesting tool 201. The harvesting tool 201 includes a shaft 202 and a head 203. The shaft 202 terminates in the head 203. The harvesting tool 201 can be driven (e.g., translated along its long axis, or moved in another manner) by the translator 204. In the example shown in FIG. 2B, the sampling probe 210 may include a grip for use as a handheld sampling probe. In some instances, the shaft 202 of the harvesting tool 201 may be directly or indirectly connected to the translator 204. In some implementations, the translator 204 can be used to secure the harvesting tool 201 inside the sampling probe 210. As shown in FIG. 2A, the sampling probe 210 is in a retracted position, in which the head 203 resides inside the tubular member 206. In some aspects of operation, the translator 204 extends the harvesting tool 201 from the sampling probe 210 through an opening at one end 214 of the sampling probe 210, for example, when performing a sampling procedure. The harvesting tool 201 may stop at an extended position where the head 203 and at least a portion of the shaft 202 reside outside of the sampling probe 210 (or tubular member 206). In some instances, the harvesting tool 201 may be ejected from the sampling probe 210 by disconnecting from the translator 204 or in another manner, which allows a reuse or recycling of the shell 206.

In some instances, the sampling probe 210 may be designed for a specific type of sampling procedure, or the sampling probe 210 may be designed with a size and shape that allows the sampling probe 210 to be used for one or more of several types of sampling procedures. For instance, operating the sampling probe 210 for specimen collection may include performing a nasal sampling, nasopharyngeal sampling, oropharyngeal sampling, throat sampling, and another type of sampling procedure. Accordingly, the samples obtained by the sampling probe 210 may include nasal samples (containing material from a nasal passage), nasopharyngeal samples (containing material from the nasopharynx), oropharyngeal samples (containing material from the oropharynx), throat samples (containing material from the throat) or other types of samples. In some instances, the harvesting tool 201 includes a common, low-cost medical grade, sterile cotton swab or another type of harvesting tool. In some instances, the harvesting tool 201 may be implemented as the harvesting tool 1000 as shown in FIG. 10 or another type of harvesting tool that has other types of features and components.

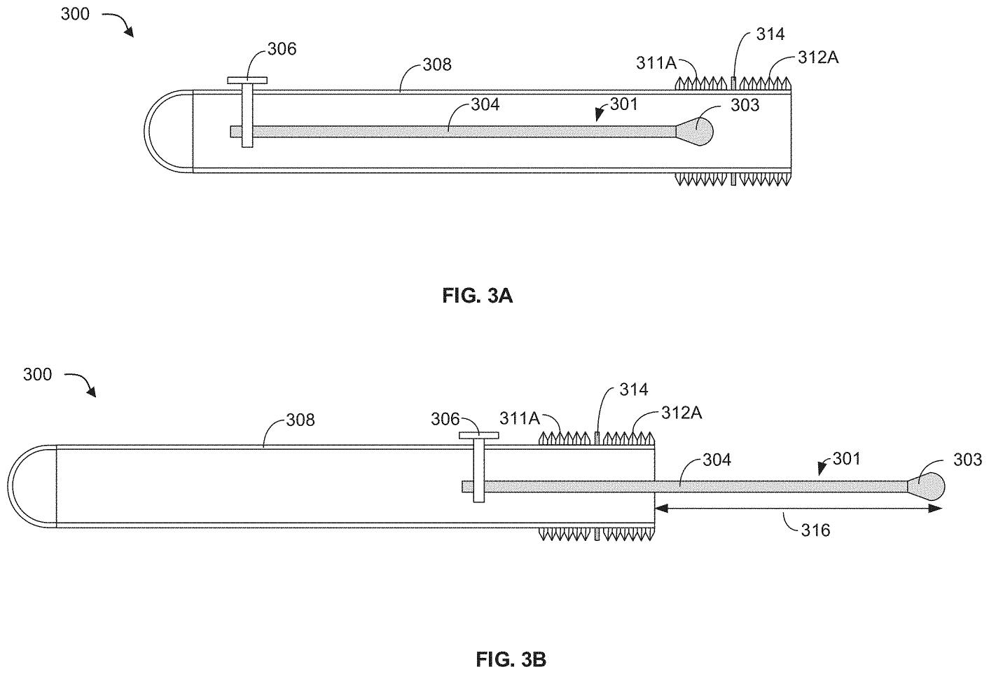

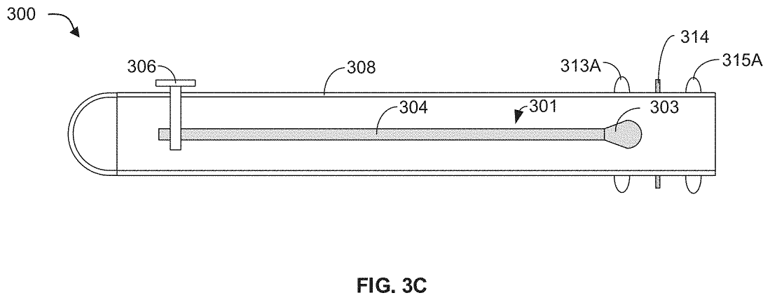

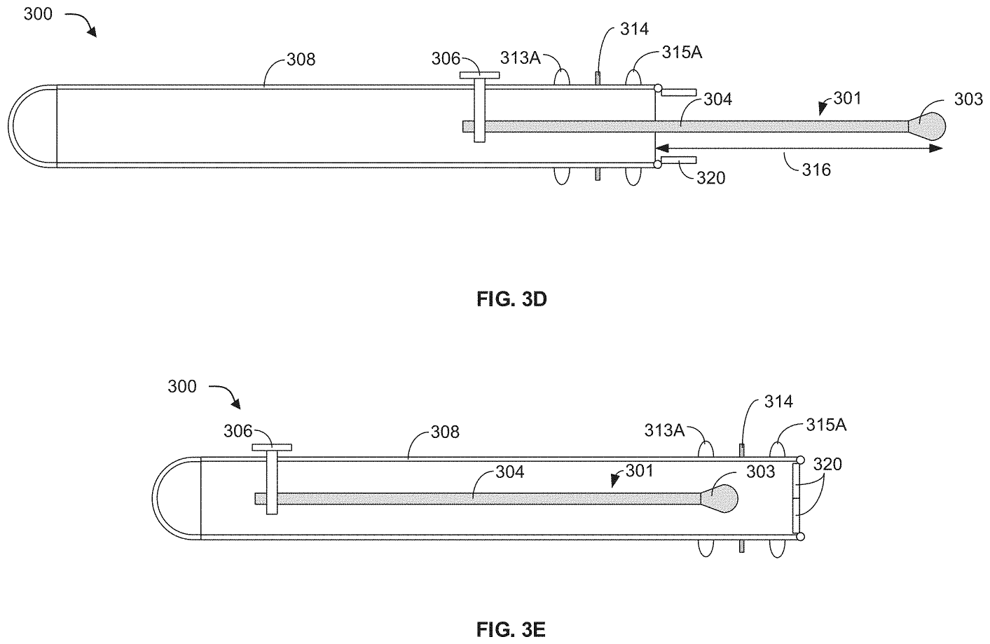

In some implementations, the translator 204 may include a spring, a push knob, or another type of component capable of extending and retracting. In some instances, the translator 204 may be configured to impart a linear motion to the harvesting tool 201 along the main axis (the long axis) of the shaft 202. In some instances, the translator 204 may also provide a spiral motion to the harvesting tool 201 (e.g., rotating the harvesting tool 201 about the main axis of the shaft 202), which can increase sample collection from the target surface. In some examples, the translator 204 may be used to retract the harvesting tool 201 into tubular member 206 after sample collection, and the interface adaptor 230 may be connected to the sampling probe 210 to prevent contamination. In some instances, the translator 204 may include a sealing unit to isolate the sample from the surrounding environment during operation and during transportation. In some instances, the translator 204 may be implemented as shown in FIGS. 3-9 or in another manner. In some instances, the translator 204 may be omitted.

In the example shown in FIGS. 2A-2B, the sampling probe 210 includes joints 211A and 212A to mechanically couple the sampling probe 210 with other components. The example joint 211A mates with a joint 211B on the housing 240, and the example joint 212A mates with a joint 212B on the interface adaptor 230 or the nasal pillow adaptor 250. In some instances, the joints 211A and 212A may be implemented as external threads as shown in FIG. 2B, snap rings, or other types of integral joints or fasteners. In some instances, the joints 211A and 212A are separated by an annular member 208. As shown in FIG. 2B, the annular member 208, which resides between the joints 211A, 212A on the outer surface of the tubular member 206, is a ring-shaped structure that is concentric with the tubular member 206. The annular member 208 may be used as a joint stopper to hold the interface adaptor 230, the nasal pillow adaptor 250, and the housing 240 at their respective positions when assembled on the sampling probe 210, as shown in FIG. 2A.

As shown in FIG. 2C, the interface adaptor 230 includes a shell 232, a joint 212B, a membrane 234, and another joint 236. In some instances, the joint 212B may be implemented as an internal thread, an external thread, a snap ring, or may be implemented in another manner to establish a mechanical connection to the joint 212A of the sampling probe 210. FIG. 2C depicts the joint 212B as an internal thread. In some instances, the shell 232, the membrane 234 and the joint 236 may be implemented as the second portion of the tubular member 112, the membrane 114 and the interface 116, respectively, as shown in FIG. 1 or in another manner.

Although FIG. 2C depicts the joints 212B, 236 as being threaded joints, non-threaded joints are possible for the joints 212B, 236. For example, threading maybe omitted to allow the joints 211A-B, 212A-B, and 236 to be slip-fit or friction-fit joints. FIG. 2D presents a schematic diagram of the example interface adapter 230 of FIG. 2C, but in which the joint 236 has unthreaded walls. Such unthreaded walls may be internally smooth and configured to slip over the mating walls of a testing instrument. Alternatively, the unthreaded walls may be externally smooth and configured to slip within the mating walls of the test instrument (e.g., within an orifice or hole). When slipped over or within the mating walls, friction may secure the contacting walls to each other (e.g., resist sliding or rotational motion) and may also form a seal therebetween. Although FIG. 2D depicts the joint 212B as being threaded, this joint may also be a slip-fit or friction-fit joint in some variations.

As shown in FIG. 2E, the housing 240 includes a shell 242 having a closed end and an open end. The shell 242 may include a joint 211B proximate the open end. In some implementations, the shell 242 is sized and shaped to fully enclose the sampling probe 210 and to provide mechanical support and concealment for samples. In some instances, the joint 211B may be implemented as an internal thread, a snap ring, or another type of integral joint or fastener to mechanically join the joint 211A with the associated joint 211B of the sampling probe 210.

In some cases, the nasal pillow adaptor 250 may be connected to the sampling probe 210 prior to the sampling process. The nasal pillow adapter 250 may serve as a sealing member at the open end of the shell that is configured to contact a subject surface (e.g., a nostril surface) and create an enclosed sampling volume when collecting a specimen. As shown in FIG. 2F, the nasal pillow adaptor 250 includes a shell 252, a nasal pillow 254 and a joint 212B. The nasal pillow adaptor 250 can form a comfortable contact and seal around the base of a nostril. The nasal pillow adaptor 250 may be disconnected and replaced by the interface adaptor 240 after sampling. In some instances, the nasal pillow 254 may include materials such as silicone, or another material. In some instances, the nasal pillow 254 may be designed with different geometry/size for patients in different age groups or for performing different sampling procedures. In some instances, the nasal pillow 254 may have a predetermined shape (e.g., with angles relative to the subject) to guide the use of the sampling probe for self-administered home sample collection.

Other types of sealing members are possible. In some implementations, the example apparatus 200 may include an oral adapter, which can form a comfortable contact and seal around a mouth. The oral adapter may have a pre-determined shape/geometry to guide the use of the sampling probe for self-administered home sample collection. For example, the oral adapter may have a threaded joint and oral pillow on opposite ends that are connected through a transition element. The transition element may be configured to allow the oral pillow to conform to a shape of the mouth (e.g., a perimeter defined by lips of the mouth). The transition element is defined by walls that connect the threaded joint to the oral pillow. In some variations, the oral adapter includes a guide operable to direct the harvesting tool 201 to a sample surface of the subject (e.g., an interior check surface, an interior throat surface, etc.). For instance, the transition element may include a hole in one of its walls and the guide may be a tube extending a passage formed by the hole along a direction that points to the sample surface. Other types of guides are possible.

In some implementations, the sampling probe 210 and the interface adaptor 230 may be composed of materials, such as synthetic polymers that are biologically compatible and resistant to chemicals used in the sampling and testing procedures. For example, the materials for the sampling probe 210 may be compatible with a variety of liquid solvents (e.g., polar or non-polar) that are used for extracting molecules from the specimen and carrying the specimen/molecules to the testing instrument.

In some instances, the sampling probe 210 is fully self-contained with the harvesting tool 210 inside the shell 206 before and after performing the sampling procedure 260 without exposing to the environment outside of the subject. As shown in FIG. 2G, the sampling probe 210 may be positioned at the base of the nostril or lip/tongue of the subject and the harvesting tool may be extended out of the shell of the sampling probe to the target surface (e.g., nasopharynx 262 and throat 264) to perform the sampling procedure. After performing the sampling procedure, the harvesting tool may be retracted back into the shell of the sampling probe and then the sampling probe may be then removed from the subject. In some instances, the shell of the sampling probe may then be capped before being removed from the subject, e.g., the hinged lid 320 as shown in FIGS. 3D and 3E.

Now referring to FIG. 2H, a schematic diagram is presented of a portion of the sampling probe 210 of FIG. 2B, but in which part of the interface adapter 230 of FIG. 2C is integrated into the example sampling probe 210. In particular, the membrane 234 and the joint 236 of the interface adapter 230 have replaced the joints 211A, 212A and annular member 208 at the end 214 of the sampling probe 210. The schematic diagram of FIG. 2H also shows a testing adapter 270 configured at first end 272 to couple to the joint 236 of the sampling probe 210 and at a second end 274 to couple to a testing instrument. FIG. 2I shows a top view of the testing adapter 270 of FIG. 2H. The first end 272 may include external threading that mates with the internal threading of the joint 236. However, other types of joints are possible between the joint 236 and the first end 272 (e.g., a snap ring, a slip fit, a friction fit, an O-ring seal, etc.). The testing adapter 270 includes a perforating element 276 configured to puncture the membrane 234 of the sampling probe 210. For example, the perforating element 276 may be a tubular structure that terminates in a sharp annular edge. The tubular structure may be suspended within the walls of the testing adapter 270 by a mount 278 configured to direct fluid flow through an interior of the tubular structure. In some variations, the perforating element 276 and the mount 278 may be integral to each other. In some variations, the perforating element 276 and the mount 278 may be integral to the testing adaptor 270.

In operation, the testing adapter 270 may be coupled to the testing instrument to facilitate sample extraction from the sampling probe 210. When coupled, the testing adapter 270 may receive solvent from the testing instrument for eluting sample material that resides on the head 203. The solvent (or a portion thereof) may be stored in a chamber 280 of the testing adapter 270. The sampling probe 210 may be then coupled to the testing adapter 270 after a sampling procedure has been completed, such as described in relation to FIG. 2G. For example, the joint 236 of the sampling probe 210 may be pressed onto the first end 272 of the testing adapter 270 and rotated to allow complementary threads to engage. Such engagement may allow a seal to form between the sampling probe 210 and the testing adapter 270. During coupling, the perforating element 276 presses against the membrane 234 and punctures the membrane 234. A passage forms through the membrane 234 that allows solvent to access and contact the head 203. In some variations, the passage is defined by the perforating element 276. Pressure may next be applied to the solvent in the chamber 280 to induce the solvent to flow past through the perforating element 276 and contact the head 203. Such contact may elute sample material from the head 203 (e.g., by dissolution, dislodging material, etc.). The eluted sample material may then be directed to the testing instrument by reversing the flow of the solvent (e.g., by suction).

FIGS. 3-9 are schematic diagrams of example sampling probes 300, 400, 500, 600, 700, 800, and 900 that can be deployed in the biological sampling apparatus 200. Other types of sampling probes may be used. As shown in FIGS. 3-9, each of the example sampling probes 300, 400, 500, 600, 700, 800, and 900 can be assembled with a housing, an interface adaptor, and a nasal pillow adaptor, e.g., the housing 240, the interface adaptor 230 and the nasal pillow adaptor 250 (or oral adapter) as shown in FIGS. 2A-2F or in another manner. In some instances, the sampling probes 300, 400, 500, 600, 700, 800, and 900 can be retracted and extended using a translator. In some instances, the sampling probes 300, 400, 500, 600, 700, 800, and 900 may include additional or different components, and the components may be arranged as shown or in another manner.

As shown in FIGS. 3A-3B, the example sampling probe 300 includes a harvesting tool 301, a knob 306, and a shell 308. The harvesting tool 301 includes a shaft 304 and a head 303, and the harvesting tool 301 is connected to the knob 306 and enclosed in the shell 308. The knob can be translated linearly (e.g., along a groove or track defined in the shell 308) to extend and retract the harvesting tool 301. In some instances, the knob 306 slides (e.g., when operated by a user) from a first position (FIG. 3A) to a second position (FIG. 3B) to extend the harvesting tool 301 from the shell 308, for example, for performing a sampling procedure. In some instances, a length 316 between one end of the shell 308 and the end the head 303 at the extended position of the harvesting tool 301 is in a range of 15-18 cm or in another range (e.g., according to the type of sample). The length 316 can be controlled by the position of the knob 306 relative to the shell 308. In some instances, the knob 306 can slide (e.g., when operated by a user) from the second position to the first position to retract the harvesting tool 301 into the shell 308, for example, after performing the sampling procedure. The example sampling probe 300 includes two joints 311A, 312A and an annular member 314 which can be implemented as the joints 211A, 212A and the annular member 208 as shown in FIG. 2B or in another manner.

As shown in FIGS. 3C-3E, the example sampling probe 300 may include two snap rings 313A, 315A and one or more hinged lids 320, which can be opened when the harvesting tool 301 is extended out of the shell 308 for sampling and closed when the harvesting 301 is retracted back into the shell 308 after sampling. In some instances, the one or more hinged lids 320 may be operated (e.g., opened or closed as shown in FIGS. 3D-3E) by the knob 306 to synchronize with the motion of the harvesting tool 301.

In some implementations, the example sampling probe 300 includes a shell 308 with a closed end and an open end. An elastomeric cap is disposed over the open end to occlude the open end. The elastomeric cap is configured to un-occlude the open end when the knob 306 is translated from the first position to the second position, thereby moving the harvesting tool 301 from the retracted position to the extended position. The elastomeric cap is also configured re-occlude the open end when the knob 306 is translated from the second position to the first position, thereby moving the harvesting tool 301 from the extended position to the retracted position.

For example, the elastomeric cap may include radial slits that intersect at a common origin to define triangular flaps. The radial slits, the common origin, and the triangular flaps may be disposed over the open end of the shell 308. Moreover, the triangular flaps may be biased to occlude the open end of the shell 308. As the harvesting tool 301 moves into the extended position, the harvesting tool 301 pushes against the triangular flaps, bending them outward as the harvesting tool 301 moves through the open end. Then, when the harvesting tool 301 returns to the retracted position, the harvesting tool 301 may disengage from the triangular flaps, allowing them to unbend and re-occlude the open end. In some variations, the harvesting tool 301 is disposed within a sheath that moves along with the harvesting tool 301. The sheath may protect the harvesting tool 301 (e.g., the head 303) from contact with the triangular flaps. Displacement of the sheath, however, may be restricted to a length less than the harvesting tool 301. This restriction allows the head 303 and at least a portion of the shaft 304 to exit the sheath before the harvesting tool 301 reaches the extended position. In some instances, the sheath is a tubular structure in which the harvesting tool 301 is nested. The tubular structure may have an open end through which the harvesting tool 301 may exit.

As shown in FIGS. 4A-4C, the example sampling probe 400 is configured to provide a multi-stage operation. As shown in FIG. 4A, the sampling probe 400 includes a harvesting tool 401, an assembly 406, and a shell 408. The harvesting tool 401 includes a shaft 404 and a head 403. The example sampling probe 400 also includes two joints 411A, 412A and an annular member 414 which can be implemented as the joints 211A, 212A and the annular member 208 as shown in FIG. 2B or in another manner.

As shown in FIG. 4A, the assembly 406 includes a knob 422 and a joint linkage 424. The joint linkage 424 provides a mechanical link between the knob 422 and the shaft 404 of the harvesting tool 401. In some instances, the shaft 404 may have a cylindrical shape or another shape. As shown in FIG. 4A, the outer surface of the shaft 404 includes a first portion of a rail groove 418A and a second portion of the rail groove 418B. In some instances, the first portion of the rail groove 418A has a linear shape along the axial direction of the shaft 404. In some instances, the second portion of the rail groove 418B has a spiral shape that spirals around the shaft 404. In some instances, the first and second portions of the rail groove 418A, 418B on the shaft 404 engage a track guide 416 that is connected to the shell 408 as shown in FIG. 4A. The track guide 416 may be implemented as a finger or another type of elongate structure that extends from the inner surface of the shell 408 into the rail groove 418A, 418B.

In some instances, the assembly 406, the rail groove 418A, 418B, and the track guide 416 are used in a multi-stage operation of the harvesting tool 401 during a sampling process. For example, during a first stage of operation, when the knob 422 slides from a first position (FIG. 4A) to a second position (FIG. 4B), the linear motion of the knob 422 provides a linear motion to the harvesting tool 401 as the track guide 416 moves along the first portion of the rail groove 418A. During a second stage of operation, when the knob 422 slides further from the second position (FIG. 4B) to a third position (FIG. 4C), the linear motion of the knob 422 drives a spiral motion of the harvesting tool 401 as the track guide 416 moves along the second portion of the rail groove 418B. In some instances, the second portion of the rail groove 418B may be designed to control the spiral motion of the harvesting tool 401 during the second stage. For example, the number of spiral turns and a pitch size between two neighboring spiral turns may be configured to control the spiral motion (e.g., a combination of a linear motion and a rotational motion) of the harvesting tool 401. The joint linkage 424 may be used to isolate the rotational motion of the harvesting tool 401 from the knob 422. In some instances, the harvesting tool 401 in the sampling probe 400 can be fully extended with a maximum length. In some instances, the length 420 between one end of the head 403 and one end of the shell 408 is in a range of 15-18 cm or in another range according to the types of samples.

As shown in FIGS. 5A-5B, the example sampling probe 500 provides an adjustable length that can be adjusted, for example, to any one of multiple discrete settings. As shown in FIGS. 5A-5B, the sampling probe 500 includes a harvesting tool 501, a joint linkage 504, a knob 506, a sleeve 520, and a shell 508. The harvesting tool 501 includes a shaft 502 and a head 503. The joint linkage 504 provides a mechanical link between the knob 506 and the sleeve 520. As shown, the example sampling probe 500 includes two joints 511A, 512A and an annular member 514, which can be implemented as the joints 211A, 212A and the annular member 208 as shown in FIG. 2B or in another manner.

In some instances, the outer surface of the sleeve 520 includes a first portion of a rail groove 518A and a second portion of the rail groove 518B. In some instances, the first and second portions of the rail groove 518A, 518B may be implemented as the first and second portions of the rail groove 418A, 418B as shown in FIGS. 4A-4C or in another manner. In some instances, the sleeve 520 can implemented as a tubular structure (e.g., a tube) and the shaft 502 of the harvesting tool 501 may be positioned in the sleeve 520. In some instances, the shaft 502 is mechanically connected with the sleeve 520. For example, the shaft 502 may be mechanically coupled to or decoupled from the sleeve 520 at the joint linkage 504, which can be controlled by the knob 506.

In some instances, a maximum extent of the length 526 can be adjusted by adjusting a position of the sleeve 520 relative to the harvesting tool 501. In some instances, an appropriate setting for the maximum extent of the length 526 may be determined based on the age group of a patient. For example, a longer length may be specified for adults, and a shorter length may be specified for children. In some instances, a position of the sleeve 520 relative to the harvesting tool 501 can be adjusted by adjusting a position of the knob 506 along the axial direction on a track 522 defined in the shell 508. In some instances, before extending the harvesting tool 501 for sampling, a mechanical coupling between the joint linkage 504 and the harvesting tool 501 may be released to allow a free motion of the sleeve 520 along the axial direction by adjusting the position of the knob 506 on the track 522. In some instances, positions of the knob 506 on the track 522 may be marked with labels 524. For example, the labels 524 may indicate age groups, e.g., “Adult” or “Child” as shown in FIGS. 5A-5B. In certain cases, the labels 524 may indicate the length 526.

In some instances, after the appropriate maximum extent for the length and/or the appropriate position of the knob 506 is determined (e.g., FIG. 5A for adult and FIG. 5B for child), the harvesting tool 501 may then engage the sleeve 520 via the joint linkage 504. In some instances, the rail grooves 518A, 518B on the sleeve 520 and a track guide 516 may be implemented as the rail grooves 418A, 418B and the track guide 416 as shown in FIGS. 4A-4C or in another manner. In some instances, the knob 506 (e.g., when operated by a user) slides along the track 522. A linear motion of the knob 506 along the track 522 may first provide a linear motion and may then provide a spiral motion to the harvesting tool 501 via the sleeve 520 as the track guide 516 moves along the first and second portions of the rail groove 518A, 518B. The joint linkage 504 can be used to isolate the rotational motion of the sleeve 520 from the knob 506.

As shown in FIGS. 6A-6B, the example sampling probe 600 includes a harvesting tool 601, a plunger seal 604, a bellow seal 606 and a shell 608. The harvesting tool 601 includes a shaft 602 and a head 603. The example sampling probe 600 includes two joints 611A, 612A and an annular member 614 which can be implemented as the joints 211A, 212A and the annular member 208 as shown in FIG. 2B or in another manner. As shown in FIGS. 6A-6B, one end of the shaft 602 is attached to a first surface of the plunger seal 604. A plunger shaft 620 is attached to a second, opposite surface of the plunger seal 604. In some instances, the plunger shaft 620 may be operated by a user to provide a linear motion to the plunger seal 604 to retract (FIG. 6A) or extend (FIG. 6B) the harvesting tool 601 along the axial direction into or out of the shell 608, respectively. In some instances, the bellow seal 606 with one end anchored on the inner surface of the shell 608 and one end on the plunger seal 604 compresses and expands as the plunger seal 604 moves in the shell 608. In some instances, the bellow seal 606 can provide an air-tight seal to the shell 608 to prevent leakage or contamination, for example, via gaps between the plunger seal 604 and the inner surface of the shell 608.

As shown in FIG. 7, the example sampling probe 700 includes a harvesting tool 701, a plunger seal 704, a bag seal 706 and a shell 708. The harvesting tool 701 includes a shaft 702 and a head 703. The example sampling probe 700 also includes two joints 711A, 712A and an annular member 714 which can be implemented as the joints 211A, 212A and the annular member 208 as shown in FIG. 2B or in another manner. As shown in FIG. 7, one end of the shaft 702 is attached to a first surface of the plunger seal 704. A plunger shaft 720 is attached to a second, opposite surface of the plunger seal 704. In some instances, the plunger shaft 720 may be operated by a user to provide a linear motion to the plunger seal 704 to extend or retract the harvesting tool 701 along the axial direction out of or into the shell 708. In some instances, the bag seal 706 with one end anchored on the inner surface of the shell 708 and one end on the plunger seal 704 compresses and expands with the movement of the plunger seal 704. In some instances, the bag seal 706 can provide an air-tight seal to the shell 708, e.g., preventing leakage or contamination, for example, via gaps between the plunger seal 704 and the inner surface of the shell 708.

As shown in FIGS. 8A-8C, the example sampling probe 800 is configured for achieving an increased (e.g., maximal) specimen collection from a target surface of a subject. As shown in FIGS. 8A-8C, the sampling probe 800 includes a harvesting tool 801, a multi-bar linkage 804, a knob 806, and a shell 808. The harvesting tool 801 includes a shaft 802 and a head 803. In some instances, the shaft 802 may be hollow, e.g., a tube or another hollow structure. The shaft 802 may include a sharp end 805, e.g., a 14-gauge needle. In some instances, the harvesting tool 801 may be implemented as the harvesting tool 1000 as shown in FIG. 10 or another type of harvesting tool. The example sampling probe 800 also includes two joints 811A, 812A and an annular member 814 which can be implemented as the joints 211A, 212A and the annular member 208 as shown in FIG. 2B or in another manner. In some instances, the knob 806 may slide along a track defined as a slot in the shell 808, e.g., similar to the track 522 shown in FIGS. 5A, 5B. For example, the knob 806 (e.g., when operated by a user) may slide between a first position shown in FIG. 8A and a second position shown in FIG. 8B along the track to extend the harvesting tool 801 for performing the sampling procedure and to retract the harvesting tool 801 after the sampling procedure.

As shown in FIGS. 8A-8C, the sampling probe 800 further includes a chamber 816. In some instances, the chamber 816 is a negative-pressure chamber sealed by a membrane 818. For example, the pressure in the chamber 816 may be less than the atmospheric pressure where the sampling probe 800 is operated. In some instances, the chamber may be sized and shaped according to the size and shape of the shell 808, the amount of sample required for testing and pressure required in the chamber. In some instances, the chamber may be sized and shaped according to other considerations. In some instances, the chamber 816 is configured adjacent to and separated from the sharp end 805 of the shaft 802, e.g., at positions as shown in FIGS. 8A-8B. In some instances, the knob 806 is further connected to a body of the chamber 816 by the multi-bar linkage 804 or in another manner. When the knob 806 is operated by a user to extend the harvesting tool 801 from a retracted position (FIG. 8A) to an extended position (FIG. 8B), the chamber 816 is extended together with the harvesting tool 801. In some implementations, the harvesting tool 801 can be fluidically coupled to the chamber 816 by puncturing the sharp end 805 of the shaft 802 through the membrane 818. In some instances, the chamber 816 may be pushed against the sharp end 805 of the shaft 802 by operating the knob 806 as shown in FIG. 8C or in another manner. In some instances, the low pressure in the chamber 816 may facilitate and maximize the amount of specimen drawn from the target surface of the subject via the head 803 which is located on an end of the shaft 802 opposite to the sharp end 805. In some instances, the specimens may be sucked into internal channels of the head 803 (e.g., the channels 1032 shown in FIG. 10G), the shaft 802, or the chamber 816. In some instances, the chamber 816 may be disengaged from the sharp end 805 of the shaft 802 before retracting the harvesting tool 801 back into the shell 808 after the sampling procedure. In some instances, a length 820 between one end of the head 803 and one end of the shell 808 is in a range of 15-18 cm or in another range (e.g., according to the types of samples).

As shown in FIGS. 9A-9C, the example sampling probe 900 is configured for achieving an increased (e.g., maximal) specimen collection from a target surface of a subject. As shown in FIGS. 9A-9C, the sampling probe 900 includes a harvesting tool 901, a joint linkage 904, a knob 906, a sleeve 920, and a shell 908. In some instances, the outer surface of the sleeve 920 includes a first portion of a rail groove 918A and a second portion of the rail groove 918B. In some instances, the first and second portions of the rail groove 918A, 918B may be implemented as the first and second portions of the rail groove 418A, 418B as shown in FIGS. 4A-4C or in another manner. In some instances, the first and second portions of the rail groove 918A, 918B on the sleeve 920 engage a track guide 916 as shown in FIGS. 9A-9C. In some instances, the joint linkage 904, the knob 906 and the sleeve 920 may be implemented and operated as the joint linkage 904, the knob 506, and the sleeve 920 as shown in FIGS. 5A-5B. In some instances, the sampling probe 900 includes two joints 911A, 912A and an annular member 914 which can be implemented as the joints 211A, 212A and the annular member 208 as shown in FIG. 2B or in another manner.

As shown in FIGS. 9A-9C, the harvesting tool 901 includes a shaft 902 and a head 922. The shaft 902 terminates in the head 922. In some instances, the head 922 may include multiple fingers 924. In some examples, each of the fingers 924 may include flock fibers, cotton fibers, polyester fibers, and woven microfibers to enhance specimen collection. In some instances, each of the fingers 924 may be implemented as the head 1004 as shown in FIGS. 10A-10G. In some instances, the fingers 924 may be extended along the radial direction as shown in FIG. 9C, which can be produced by a spiral motion of the shaft 902 when the track guide 916 moves along the second portion of the rail grooves 918B on the sleeve 920. In certain examples, the fingers 924 may be extended along the radial direction in another manner. In some implementations, the radially extended fingers 924 with a spiral motion may provide additional contact to better collect cells, molecules, or microorganisms from the target surface.

FIGS. 10A-10D are schematic diagrams showing aspects of an example harvesting tool 1000. In the examples shown, the harvesting tool 1000 includes a shaft 1002 and a head 1004 mounted on one end of the shaft 1002. In some instances, the shaft 1002 may be composed of plastic, wood, or another material. In some instances, the head 1004 has a cylindrical shape. In some instances, the head 1004 includes materials that are chemical resistance, and mechanically stable, for example silicone, polydimethylsiloxane (PDMS), polyether ether ketone (PEEK), polysulfone (PS) or another material. In some instances, the harvesting tool 1000 may be a monolithic unit. In some instances, the head 1004 may have a diameter of 3.0 mm and a length less than 12.7 mm. In some instances, the head 1004 may have another geometry or dimensions in another range. In some instances, the harvesting tool 1000 may be injection molded or manufactured in another manner. In some examples, the harvesting tool 1000 may include additional or different components, and the components may be arranged as shown or in another manner.

The example head 1004 shown in FIG. 10A has a specimen acquisition surface 1006. As shown in FIG. 10A, the specimen acquisition surface 1006 includes an array of specimen acquisition regions 1010, each of which is configured to acquire and retain a respective volume of liquid sample. In some instances, each of the specimen acquisition regions 1010 is circumferential arranged on the specimen acquisition surface 1006. The specimen acquisition regions 1010 can all be oriented along the same direction (e.g., a clockwise direction or a counterclockwise direction) and parallel to each other. In certain instances, the specimen acquisition regions 1010 on the specimen acquisition surface 1006 may be arranged and oriented in another manner.

As shown in FIGS. 10C-10F, each of the specimen acquisition regions 1010 may be fluidically isolated from neighboring specimen acquisition regions 1010. In some instances, each of the specimen acquisition regions 1010 may have an oval shape (as shown in FIG. 10C), a rectangular shape, or another shape. The specimen acquisition region 1010 may include a containment chamber 1020 with a shallow region 1022, e.g., a groove, a dent, or a trench, and a deep region 1024, e.g., a hole. In some instances, the specimen acquisition region 1010 may include a combination of shallow and deep regions 1022, 1024. In some instances, the shallow and deep regions 1022, 1024 in a containment chamber 1020 may be arranged as shown in FIGS. 10D-10F or in another manner. In some instances, each of the specimen acquisition regions 1010 may have inner surfaces coated with flock fibers or another type of material to prevent dripping of samples collected in the specimen acquisition regions 1010. As shown in FIG. 10F, the specimen acquisition region 1010 may include a descending cut 1026 which allows an efficient collection of specimen from the target surface and retainment of the specimen in the deep region 1024.

In some instances, the specimen acquisition regions 1010 on the head 1004 can be inter-connected. For example, as shown in FIG. 10G, each of the specimen acquisition regions 1010 can be fluidically coupled with an interior volume of the shaft 1002 (e.g., a hollow shaft) via respective channels 1032. During operation, liquid samples from a target surface may be collected into at least one of the specimen acquisition regions 1010 and extracted via the respective channel 1032 to the interior volume of shaft 1002, which can act as a conduit to communicate fluid from the head 1004 (e.g., to a collection chamber or another type of container or region). In some instances, the inner surface of the specimen acquisition regions 1010, the channels 1032 and the shaft 1002 can be coated with hydrophobic materials for maximal sample extraction. In some instances, the harvesting tool 1000 with the inter-connected specimen acquisition regions 1010 may be integrated in a sampling probe which includes a negative-pressure chamber (e.g., the sampling probe 800 with the chamber 816).

In a general aspect of what is described above, a biological sampling apparatus can be used to obtain a sample (e.g., a swab sample or another type of biological specimen).

In a first example, a biological sampling apparatus includes a sampling probe and a housing. The sampling probe includes a harvesting tool, a translator and a shell. The translator can extend a portion of the harvesting tool from the shell (e.g., to perform a sampling procedure to obtain a sample), and can retract the harvesting tool into the shell (e.g., to secure the sample after the sampling procedure).

Implementations of the first example may include one or more of the following features. The harvesting tool can be a swab that includes a swab shaft and a swab head. The biological sampling apparatus may include an interface. The interface can connect with an interface adapter. The interface or the interface adapter can include a membrane. The interface or the interface adaptor can be configured to attach to the sampling probe and to a testing instrument (e.g., to test the sample). The biological sampling apparatus may include any combination of features shown in, or described with respect to, FIGS. 1A, 1B, 2A, 2B, 2C, 2D, 2E, and 2F, as well as other types of features. The sampling probe may include any combination of features shown in, or described with respect to, FIGS. 3A, 3B, 4A, 4B, 4C, 5A, 5B, 6A, 6B, 7, 8A, 8B, 8C, 9A, 9B, 9C, 10A, 10B, 10C, 10D, 10E, 10F, 10G, as well as other types of features.

Implementations of the first example may include one or more of the following features. The translator can include a knob and a track. The translator can be configured to provide a spiral motion to the harvesting tool during the sampling procedure. The harvesting tool can include a shaft with a rail groove. The rail groove can include a spiral portion. The translator can include a track guide engaging the rail groove. The translator can include a knob and a sleeve. The shaft of the harvesting tool can be mechanically connected to or disconnected from the sleeve via a joint linkage associated with the knob. The translator can include a plunger seal. The translator can include a bellow seal or a bag seal to prevent contamination to the sample. The sampling probe can include a negative-pressure chamber. The negative-pressure chamber can include a membrane. The harvesting tool can include a hollow shaft fluidically coupled to the head. The negative-pressure chamber can be activated by a linkage mechanism to drive a sharp end of the hollow shaft to puncture the membrane and to extract a liquid sample from the head. The harvesting tool can include a head with radially extendable fingers. The harvesting tool includes a head with a plurality of specimen acquisition regions. Each of the plurality of specimen acquisition regions can include a containment chamber for collecting and retaining a liquid sample.

In some aspects of what is described, a biological sampling apparatus may be described by the following examples:

### Example 1

A biological sampling apparatus comprising:
a shell having a closed end and an open end;
a harvesting tool comprising a shaft and a head, the shaft terminating in the head;
a translator configured to move the harvesting tool through the open end between a retracted position, where the head resides in the shell, and an extended position, where the head and at least a portion of the shaft reside outside the shell; and
a sealing member at the open end configured to contact a subject surface and to create an enclosed sampling volume when collecting a specimen.

### Example 2

The apparatus of example 1, comprising a covering at the open end of the shell that forms an enclosed volume with the shell to isolate the head from an external environment of the apparatus when the translator is in the retracted position.

### Example 3

The apparatus of example 2, wherein the covering comprises a hinged lid configured to:
open when the translator moves the head from the retracted position to the extended position; and
close when the translator moves the head from the extended position to the retracted position.

### Example 4

The apparatus of example 2, wherein the covering comprises a membrane.

### Example 5

The apparatus of example 1 or any one of examples 2-4, wherein the translator is configured to rotate the harvesting tool when moving between the retracted and extended positions.

### Example 6

The apparatus of example 1 or any one of examples 2-4, comprising a sampling adapter coupled to the open end of the shell and comprising the sealing member.

### Example 7

The apparatus of example 1 or any one of examples 2-4, wherein the sealing member comprises a nasal pillow configured to contact a surface about a nasal passage; and
wherein the enclosed sampling volume includes the nasal passage.

### Example 8

The apparatus of example 1 or any one of examples 2-4, wherein the head comprises a plurality of fingers.

### Example 9

The apparatus of example 1 or any one of examples 2-4, wherein the head comprises a specimen acquisition surface having a plurality of specimen acquisition regions.

### Example 10

The apparatus of example 9, wherein the shaft comprises a hollow shaft fluidically coupled to each of the plurality of specimen acquisition regions.

### Example 11

The apparatus of example 1 or any one of examples 2-4, wherein the head comprises a swab.

In some aspects of what is described, a biological sampling method may be described by the following examples:

### Example 1

A biological sampling method comprising:
obtaining a biological sampling apparatus comprising:
   a shell having a closed end and an open end,
   a harvesting tool comprising a shaft and a head, the shaft terminating in the head,
   a translator configured to move the harvesting tool, and
   a sealing member at the open end of the shell;
positioning the sealing member in contact with a surface of a subject to enclose a sampling volume;
moving the translator from a retracted position, where the head resides in the shell, to an extended position, where the head and at least a portion of the shaft reside outside the shell and in the enclosed sampling volume;
collecting a specimen from the enclosed sampling volume; and
moving the head from the second position to the first position while the sealing member remains in contact with the subject surface.

### Example 2

The method of example 1, comprising:
opening a covering at the open end of the shell when the translator moves from the retracted position to the extended position; and
closing the covering when the translator moves from the extended position to the retracted position.

### Example 3

The method of example 1 or example 2, wherein a sampling adapter is
coupled to the open end of the shell and comprises the sealing member.

### Example 4

The method of example 3, comprising:
removing the sampling adapter from the open end of the shell; and
coupling a testing adapter to the open end of the shell, the testing adapter configured to interface with a testing instrument.

### Example 5

The method of example 1 or example 2, comprising rotating the head while collecting the specimen.

### Example 6

The method of example 1 or example 2,
wherein the sealing member comprises a nasal pillow configured to contact the surface of the subject about a nasal passage, and
wherein the enclosed sampling volume comprises the nasal passage.

### Example 7

The method of example 1 or example 2, wherein the head comprises a swab.

### Example 8

The method of example 1, wherein the subject performs the operations of obtaining the biological sampling apparatus, positioning the sealing member, moving the translator, collecting the specimen, and moving the head.

### Example 9

The method of example 2, wherein the subject performs the operations of obtaining the biological sampling apparatus, positioning the sealing member, moving the translator, collecting the specimen, moving the head, opening a covering, and closing the covering.

### Example 10

The method of example 1 or example 2, wherein collecting the specimen comprises collecting a COVID-19 specimen from the enclosed sampling volume.

### Example 11

The method of example 10, wherein the COVID-19 specimen comprises a COVID-19 virus or portion thereof.

### Example 12

The method of example 10, wherein the COVID-19 specimen comprises biological material suspected of containing a COVID-19 virus or portion thereof.

### Example 13

The method of example 10, wherein the COVID-19 specimen comprises biological material indicating an infection, past or present, of the subject by COVID-19.

### Example 14

The method of example 13, wherein the biological material comprises antibodies produced by the subject in response to the infection by COVID-19.

### Example 15

The method of example 13, wherein the biological material comprises ribonucleic acid (RNA) material of a COVID-19 virus.

In some aspects of what is described, a biological sampling apparatus may be additionally be described by the following examples:

### Example 1

A biological sampling apparatus comprising
a shell having a closed end and an open end;
a harvesting tool comprising a shaft and a head, the shaft terminating in the head;
a translator configured to move the harvesting tool through the open end between a retracted position, where the head resides in the shell, and an extended position, where the head and at least a portion of the shaft reside outside the shell; and
a testing adapter comprising a first open end and a second open end, the first open end configured to couple to the open end of the shell, the second open end configured to couple with a testing instrument.

### Example 2

The apparatus of example 1, comprising a covering at the open end of the shell that forms an enclosed volume with the shell to isolate the head from an external environment of the apparatus when the translator is in the retracted position.

### Example 3

The apparatus of example 2, wherein testing adapter comprises a perforating element configured to puncture the covering when the first open end of the testing adapter couples to the open end of the shell.

### Example 4

The apparatus of example 3, wherein the perforating element is a tubular structure that terminates in a sharp annular edge.

### Example 5

The apparatus of example 1 or any one of examples 2-4, wherein the testing adapter comprises a covering that forms an enclosed volume with the shell to isolate the head from an external environment of the apparatus when the translator is in the retracted position.

### Example 6

The apparatus of example 7, wherein the covering comprises a membrane.

While this specification contains many details, these should not be understood as limitations on the scope of what may be claimed, but rather as descriptions of features specific to particular examples. Certain features that are described in this specification or shown in the drawings in the context of separate implementations can also be combined. Conversely, various features that are described or shown in the context of a single implementation can also be implemented in multiple implementations separately or in any suitable sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single product or packaged into multiple products.

A number of embodiments have been described. Nevertheless, it will be understood that various modifications can be made. Accordingly, other embodiments are within the scope of the present disclosure.

1. A biological sampling apparatus comprising:
a shell having a closed end and an open end;
a harvesting tool comprising a shaft and a head, the shaft terminating in the head;
a translator configured to move the harvesting tool through the open end between a retracted position, where the head resides in the shell, and an extended position, where the head and at least a portion of the shaft reside outside the shell;
a covering at the open end of the shell that forms an enclosed volume with the shell to isolate the head from an external environment of the apparatus when the translator is in the retracted position; and
a sealing member at the open end configured to contact a subject surface and to create an enclosed sampling volume when collecting a specimen.

2. (canceled)

3. The apparatus of claim 0, wherein the covering comprises a hinged lid configured to:
open when the translator moves the head from the retracted position to the extended position; and
close when the translator moves the head from the extended position to the retracted position.

4. The apparatus of claim 1, wherein the covering comprises a membrane.

5. The apparatus of claim 1, wherein the translator is configured to rotate the harvesting tool when moving between the retracted and extended positions.

6. The apparatus of claim 1, comprising a sampling adapter coupled to the open end of the shell and comprising the sealing member.

7. The apparatus of claim 1,
wherein the sealing member comprises a nasal pillow configured to contact a surface about a nasal passage; and
wherein the enclosed sampling volume includes the nasal passage.

8. The apparatus of claim 0, wherein the head comprises a plurality of fingers.

9. The apparatus of claim 0, wherein the head comprises a specimen acquisition surface having a plurality of specimen acquisition regions.

10. The apparatus of claim 9, wherein the shaft comprises a hollow shaft fluidically coupled to each of the plurality of specimen acquisition regions.

11. The apparatus of claim 1, wherein the head comprises a swab.

12. A biological sampling method comprising:
obtaining a biological sampling apparatus comprising:
a shell having a closed end and an open end,
a harvesting tool comprising a shaft and a head, the shaft terminating in the head,
a translator configured to move the harvesting tool, and
a sealing member at the open end of the shell;
positioning the sealing member in contact with a surface of a subject to enclose a sampling volume;
moving the translator from a retracted position, where the head resides in the shell, to an extended position, where the head and at least a portion of the shaft reside outside the shell and in the enclosed sampling volume;
collecting a specimen from the enclosed sampling volume; and
moving the head from the second position to the first position while the sealing member remains in contact with the subject surface.

13. The method of claim 12, comprising:
opening a covering at the open end of the shell when the translator moves from the retracted position to the extended position; and
closing the covering when the translator moves from the extended position to the retracted position.

14. The method of claim 12, wherein a sampling adapter is coupled to the open end of the shell and comprises the sealing member.

15. The method of claim 14, comprising:
removing the sampling adapter from the open end of the shell; and
coupling a testing adapter to the open end of the shell, the testing adapter configured to interface with a testing instrument.

16. The method of claim 12, comprising rotating the head while collecting the specimen.

17. The method of claim 12,
wherein the sealing member comprises a nasal pillow configured to contact the surface of the subject about a nasal passage, and
wherein the enclosed sampling volume comprises the nasal passage.

18. The method of claim 12, wherein the head comprises a swab.

19. The method of claim 12, wherein the subject performs the operations of obtaining the biological sampling apparatus, positioning the sealing member, moving the translator, collecting the specimen, and moving the head.

20. The method of claim 12, wherein collecting the specimen comprises collecting a COVID-19 specimen from the enclosed sampling volume.

21. A biological sampling apparatus comprising:
a shell having a closed end and an open end;
a harvesting tool comprising a shaft and a head, the shaft terminating in the head;
a translator configured to move the harvesting tool through the open end between a retracted position, where the head resides in the shell, and an extended position, where the head and at least a portion of the shaft reside outside the shell;
a covering at the open end of the shell that forms an enclosed volume with the shell to isolate the head from an external environment of the apparatus when the translator is in the retracted position; and
a testing adapter comprising a first open end and a second open end, the first open end configured to couple to the open end of the shell, the second open end configured to couple with a testing instrument.

22. (canceled)

23. The apparatus of claim 21, wherein testing adapter comprises a perforating element configured to puncture the covering when the first open end of the testing adapter couples to the open end of the shell.

24. The apparatus of claim 23, wherein the perforating element is a tubular structure that terminates in a sharp annular edge.

25. The apparatus of claim 21, wherein the testing adapter comprises a covering that forms an enclosed volume with the shell to isolate the head from an external environment of the apparatus when the translator is in the retracted position.

26. The apparatus of claim 25, wherein the covering comprises a membrane.
